(19) United States
(12) Patent Application Publication
(10) Pub. No.: US 2025/0191181 A1
(43) Pub. Date: Jun. 12, 2025

(54) POSTURE EVALUATION APPARATUS, POSTURE EVALUATION SYSTEM, POSTURE EVALUATION METHOD, AND NON-TRANSITORY COMPUTER-READABLE MEDIUM

(71) Applicants: NEC CORPORATION, Tokyo (JP); National University Corporation Tokyo Medical and Dental University, Tokyo (JP)

(72) Inventors: Shuhei NOYORI, Tokyo (JP); Makoto YASUKAWA, Tokyo (JP); Kosuke NISHIHARA, Tokyo (JP); Yuki KOSAKA, Tokyo (JP); Akimoto NIMURA, Tokyo (JP); Koji FUJITA, Tokyo (JP); Takuya IBARA, Tokyo (JP)

(73) Assignees: NEC CORPORATION, Tokyo (JP); National University Corporation Tokyo Medical and Dental University, Tokyo (JP)

(21) Appl. No.: 18/846,505

(22) PCT Filed: Feb. 2, 2023
(86) PCT No.: PCT/JP2023/003302

§ 371 Date: Sep. 12, 2024

(30) Foreign Application Priority Data
Mar. 31, 2022 (JP) 2022-058198

(51) Int. Cl. G06T 7/00; A61B 5/00; G06T 7/70

(52) U.S. Cl. CPC G06T 7/0012; A61B 5/4561; A61B 5/742; G06T 7/70; G06T 2207/30012

(57) ABSTRACT

A posture evaluation apparatus, a posture evaluation system, a posture evaluation method, and a non-transitory computer-readable medium that are able to evaluate a posture with high accuracy at a relatively low cost are provided. A posture evaluation apparatus includes a spine extracting means for extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image, a feature value calculating means for calculating a feature value about at least a spine, based on the position information and the spine edge point cloud, and a state estimating means for estimating a state of at least the spine, based on the feature value.

### TECHNICAL FIELD

The present disclosure relates to a posture evaluation apparatus, a posture evaluation system, a posture evaluation method, and a non-transitory computer-readable medium.

### BACKGROUND ART

In recent years, with spread of online training and self-training, there is an increasing need for ordinary people without specialized knowledge to evaluate own postures.

Patent Literature 1 describes a motion evaluation system that extracts feature values from video data acquired by capturing images of a body by using a terminal carried by a user, identifies a position of each portion of the body, and displays movements of bones connecting the portions to be superimposed on the video.

### CITATION LIST

### Patent Literature

Patent Literature 1: Japanese Unexamined Patent Application Publication No. 2020-141806

### SUMMARY OF INVENTION

### Technical Problem

However, in the motion evaluation system described in Patent Literature 1, a trunk is represented by a straight line or a rectangle, and accordingly, a posture cannot be evaluated based on a spine shape itself. Therefore, there is a problem that accuracy of the posture evaluation in Patent Literature 1 is not sufficient as compared with accuracy of posture evaluation by an expert such as a therapist and a trainer. Methods for measuring the spine shape include a method using a depth camera, a method using an acceleration sensor, and a method of scanning a measuring probe along the spine, but all of the above require expensive specialized equipment. Therefore, these methods are not suitable for ordinary people to evaluate own postures.

An object of the present disclosure is to provide a posture evaluation apparatus, a posture evaluation system, a posture evaluation method, and a non-transitory computer-readable medium that are able to evaluate a posture with high accuracy at a relatively low cost.

### Solution to Problem

A posture evaluation apparatus according to the present disclosure includes: a spine extracting means for extracting, based on an image acquired by imaging a side surface of a body of a subject person and position information about at least a cervical vertebrae, hip joints, knee joints of the body on the image, a spine edge point cloud constituted of a predetermined number of points representing a spine shape on the image; a feature value calculating means for calculating a feature value about at least a spine, based on the position information and the spine edge point cloud; and a state estimating means for estimating a state of at least the spine, based on the feature value.

A posture evaluation system according to the present disclosure includes a posture evaluation apparatus, and a subject person terminal that can communicate with the posture evaluation apparatus, wherein the posture evaluation apparatus includes: a spine extracting means for extracting, based on an image of a side surface of a body of a subject person acquired by the subject person terminal and position information about at least a cervical vertebrae, hip joints, and knee joints of the body on the image, a spine edge point cloud constituted of a predetermined number of points representing a spine shape on the image; a feature value calculating means for calculating a feature value about at least a spine, based on the position information and the spine edge point cloud; and a state estimating means for estimating a state of at least the spine, based on the feature value.

A posture evaluation method according to the present disclosure is a method including, by a posture evaluation apparatus: extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image; calculating a feature value about at least a spine, based on the position information and the spine edge point cloud; and estimating a state of at least the spine, based on the feature value.

A non-transitory computer-readable medium according to the present disclosure is a non-transitory computer-readable medium configured to store a program causing a posture evaluation apparatus to execute: processing of extracting, based on an image acquired by imaging a side surface of a body of a subject person and position information about at least a cervical vertebrae, hip joints, knee joints of the body on the image, a spine edge point cloud constituted of a predetermined number of points representing a spine shape on the image; processing of calculating a feature value about at least a spine, based on the position information and the spine edge point cloud; and processing of estimating a state of at least the spine, based on the feature value.

### Advantageous Effects of Invention

It is possible to provide a posture evaluation apparatus, a posture evaluation system, a posture evaluation method, and a non-transitory computer-readable medium that are able to evaluate a posture with high accuracy at a relatively low cost.

### BRIEF DESCRIPTION OF DRAWINGS

FIG. 1 is a block diagram illustrating a configuration of a posture evaluation apparatus according to a first example embodiment;

FIG. 2 is a block diagram illustrating a configuration of a posture evaluation apparatus according to a second example embodiment;

FIG. 3 is a drawing illustrating an example of an image captured by an image-capturing unit according to the second example embodiment;

FIG. 4 is a drawing illustrating an example of key points according to the second example embodiment;

FIG. 5 is a drawing illustrating an example of a spine edge point cloud according to the second example embodiment;

FIG. 6 is a diagram for explaining processing of a spine extracting unit according to the second example embodiment;

FIG. 7 is a diagram for explaining processing of a feature value calculating unit according to the second example embodiment;

FIG. 8 is a diagram for explaining processing of the feature value calculating unit according to the second example embodiment;

FIG. 9 is a diagram for explaining processing of a state estimating unit according to the second example embodiment;

FIG. 10 is a drawing illustrating an example of an image displayed on a display unit according to the second example embodiment;

FIG. 11 is a drawing illustrating another example of an image displayed on the display unit according to the second example embodiment;

FIG. 12 is a table illustrating a data structure of a reference value list according to the second example embodiment;

FIG. 13 is a flowchart illustrating a posture evaluation method according to the second example embodiment;

FIG. 14 is a block diagram illustrating a configuration of a posture evaluation apparatus according to a third example embodiment;

FIG. 15 is a drawing illustrating another example of an image displayed on a display unit of the posture evaluation apparatus according to the third example embodiment; and

FIG. 16 is a diagram illustrating a posture evaluation system according to a fourth example embodiment.

### EXAMPLE EMBODIMENT

### First Example Embodiment

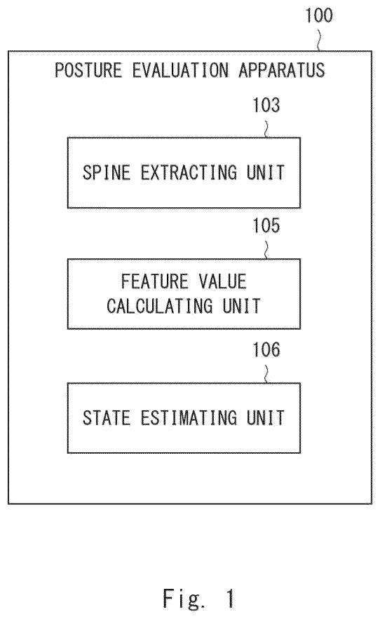

This first example embodiment is described with reference to FIG. 1. FIG. 1 is a block diagram illustrating a configuration of a posture evaluation apparatus 100 according to this first example embodiment.

The posture evaluation apparatus 100 in this first example embodiment is 10 an apparatus that evaluates posture based on an image acquired by capturing an image of a side surface of a body with a camera such as a smartphone. Specifically, the posture evaluation apparatus 100 estimates a spine shape, which is the shape of the spine in the image, and evaluates the posture based on the spine shape. This allows the posture to be evaluated at a relatively low cost with 15 a high accuracy in situations such as online training and self-training.

As illustrated in FIG. 1, the posture evaluation apparatus 100 includes a spine extracting unit 103, a feature value calculating unit 105, and a state estimating unit 106.

The spine extracting unit 103 extracts a spine edge point cloud constituted by a predetermined number of points representing the spine shape on the image based on an image acquired by imaging the side surface of the body of the subject person and position information of at least the cervical vertebrae, hip joints, and knee joints of the body on the image. It should be noted that the subject person means a person whom posture is evaluated by the posture evaluation apparatus 100.

In this case, the image acquired by capturing the image of the side surface of the body of the subject person is a two-dimensional image, and may be a two-dimensional RGB image.

The spine edge point cloud is a point cloud constituted by multiple points that represent the spine on the image. Each point that constitutes the spine edge point cloud may be a single pixel, or an image region consisting of N pixels vertically and M pixels horizontally. N and M are positive integers, and N and M may be equal or different.

The position information of the cervical vertebrae on the image is, for example, the position information of any one of the seven vertebrae that make up the cervical vertebrae, for example, the position information of the protuberance vertebra (C7). The position information of the knee joints on the image is position information of any one of the lower end of the femur, the patella, the upper end of the tibia, the upper end of the fibula, and the knee joints space, and is, for example, the position information of the lower end of the femur. Specifically, the position information of the vertebrae is, for example, the position information of a pixel located at the center of an image region corresponding to the vertebrae on the image. Similarly, the position information of the lower end of the femur is, for example, the position information of a pixel located at the center of an image region corresponding to the lower end of the femur on the image.

The position information on an image is, for example, image coordinates. In this case, the image coordinates are coordinates for indicating the position of a pixel on a two-dimensional image, and are defined as, for example, a coordinate system in which the center of the pixel located at the leftmost and uppermost side of the two-dimensional image is defined as the origin, the left-right or horizontal direction is defined as the x-direction, and the up-down or vertical direction is defined as the y-direction.

The feature value calculating unit 105 calculates a feature value for at least the spine, based on the position information of at least the cervical vertebrae, hip joints, and knee joints on the image and the spine edge point cloud.

The state estimating unit 106 estimates the state of at least the spine, based on the feature value calculated by the feature value calculating unit 105.

According to this first example embodiment, the posture evaluation apparatus 100 that can evaluate the posture with a high accuracy at a relatively low cost can be provided. Specifically, the spine extracting unit 103 extracts a spine edge point cloud that represents the spine shape on the image, and the feature value calculating unit 105 calculates the feature value of the spine based on the position information of the cervical vertebrae, hip joints, and knee joints on the image and the spine edge point cloud. Then, the state estimating unit 106 estimates the state of the spine based on the feature value. In other words, the posture can be evaluated based on the spine shape on the image, so that the posture can be evaluated with a high accuracy. Furthermore, the posture evaluation can be performed based on the spine shape without using expensive specialized equipment, so that the posture can be evaluated at a relatively low cost. Therefore, the posture evaluation apparatus 100 that can evaluate the posture with a high accuracy at a relatively low cost can be provided.

Methods for measuring the spine shape include a method using a depth camera, a method using an acceleration sensor, and a method of scanning a measuring probe along the spine, but all of them require expensive specialized equipment. These methods aim to accurately estimate the position and inclination of each of the multiple vertebrae that make up the spine. However, experts such as therapists and trainers evaluate the balance of the overall bending of the spine, and do not evaluate the position and inclination of each vertebra. Therefore, there is no need for ordinary people to aim for the same level of accuracy as these methods when evaluating their own posture at an expert level. Conversely, the posture evaluation apparatus 100 according to the first example embodiment can evaluate posture with the same high level of accuracy as experts such as therapists and trainers, based on the spine edge point cloud that represents the spine shape on the image.

Also, there is a problem that the state of the hip joints alone cannot be evaluated when the trunk is expressed as a straight line or a rectangle and the hip joints and the spine are evaluated together as in the motion evaluation system described in Patent Literature 1. Conversely, in the posture evaluation apparatus 100 according to the first example embodiment, the spine shape on the image is expressed as a spine edge point cloud, so that the state of only the hip joints can be evaluated.

### Second Example Embodiment

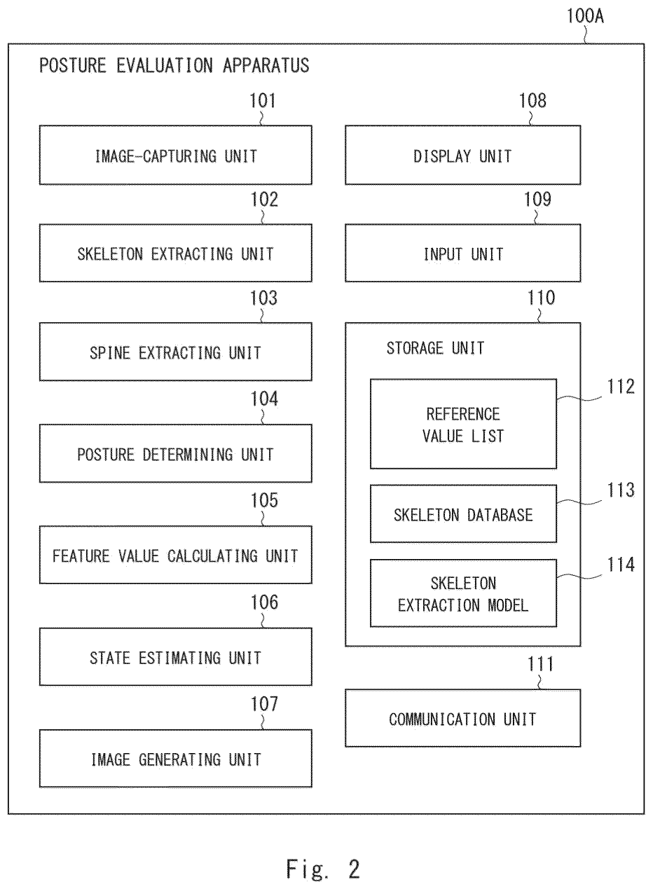

This second example embodiment is described with reference to FIG. 2. FIG. 2 is a block diagram illustrating a configuration of a posture evaluation apparatus 100A according to this second example embodiment. The posture evaluation apparatus 100A is, for example, a user terminal such as a smartphone, a tablet terminal, or a personal computer owned by a user. The user includes both a subject person whose posture is evaluated by the posture evaluation apparatus 100A, and an evaluator who uses the posture evaluation apparatus 100A to evaluate the posture of others. When a subject person uses the posture evaluation apparatus 100A to evaluate his/her own posture in self-training or the like, the subject person is also the evaluator. When an evaluator uses the posture evaluation apparatus 100A to evaluate the posture of others, the evaluator is, for example, a therapist or trainer.

As illustrated in FIG. 2, the posture evaluation apparatus 100A according to this second example embodiment includes an image-capturing unit 101, a skeleton extracting unit 102, a spine extracting unit 103, a posture determining unit 104, a feature value calculating unit 105, a state estimating unit 106, an image generating unit 107, a display unit 108, an input unit 109, a storage unit 110, and a communication unit 111. The input unit 109 and the display unit 108 may be integrated into a single touch panel display, or may be provided separately. The storage unit 110 stores a reference value list 112, a skeleton database (illustrated as a “skeleton DB” in FIG. 2) 113, a skeleton extraction model 114, and the like.

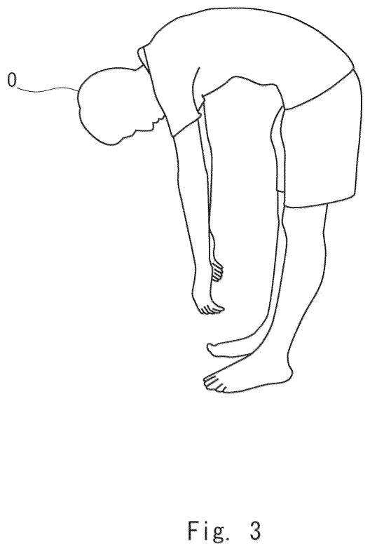

The image-capturing unit 101 captures an image of the side surface of the body of the subject person. FIG. 3 illustrates an example of an image of the side surface of the body of the subject person captured by the image-capturing unit 101. The image illustrated in FIG. 3 illustrates the side surface of a person O, which corresponds to the subject person bending forward. In this case, the image captured by the image-capturing unit 101 is a two-dimensional image, and may be a two-dimensional RGB image. The image-capturing unit 101 inputs the captured image to the skeleton extracting unit 102 and the spine extracting unit 103.

The image-capturing unit 101 may also capture a video of the side surface of the body of the subject person to acquire an image. In this case, the user may specify the time point for performing posture evaluation by operating the input unit 109. The image at the time point specified by the user may then be input to the skeleton extracting unit 102 and the spine extracting unit 103.

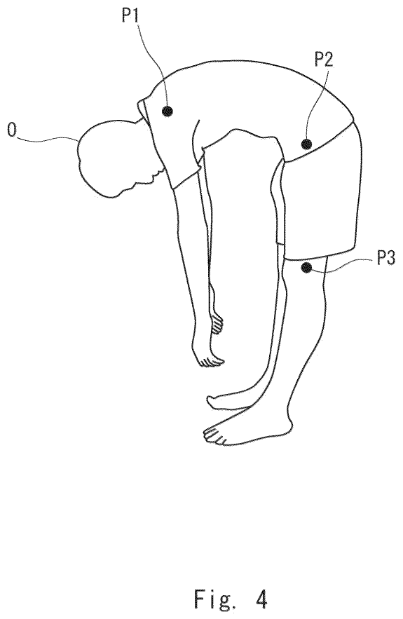

The skeleton extracting unit 102 extracts position information of at least the cervical vertebrae, hip joints, and knee joints (hereinafter, also referred to as “position information of key points”) from the image captured by the image-capturing unit 101. An example of key points P1, P2, and P3 extracted by the skeleton extracting unit 102 from the image illustrated in FIG. 3 is illustrated in FIG. 4. In FIG. 4, P1 is the key point of the vertebrae (hereafter referred to as “vertebrae key point”), P2 is the key point of the hip joints (hereafter referred to as “hip joints key point”), and P3 is the key point of the lower end of the femur (hereafter referred to as “knee joints key point”). It should be noted that the details of the position information are as described in the first example embodiment, so the explanation is omitted here.

Specifically, the skeleton extracting unit 102 extracts position information of key points from the image captured by the image-capturing unit 101 using the trained skeleton extraction model 114. The posture evaluation apparatus 100A performs machine learning in advance using the skeleton extraction model 114, i.e., a machine learning model, and the skeleton database 113, i.e., training data, to generate the trained skeleton extraction model 114.

The skeleton extracting unit 102 inputs the position information of the extracted key points to the spine extracting unit 103.

The position information of the key points may be information expressed in three-dimensional coordinates defined by the z direction, i.e., the depth direction, in addition to the x direction, i.e., the left-right or horizontal direction, and the y direction, i.e., the up-down or vertical direction, of the two-dimensional image captured by the image-capturing unit 101. This is possible by using a skeleton extraction model 114 that extracts the position information of the key points expressed in three-dimensional coordinates from the two-dimensional image.

In addition, the body parts from which the skeleton extracting unit 102 extracts key points are not limited to the above-mentioned cervical vertebrae, hip joints, and knee joints, but may extract key points from, for example, ankle joints, shoulder joints, elbow joints, and wrist joints. Furthermore, the skeleton extracting unit 102 may extract eyes, ears, center of the head, and the like, as key points in addition to joints.

In addition, the key points may be specified by displaying the image captured by the image-capturing unit 101 on the display unit 108 and the user operating the input unit 109. Then, the position information of the key points specified by the user may be input to the spine extracting unit 103.

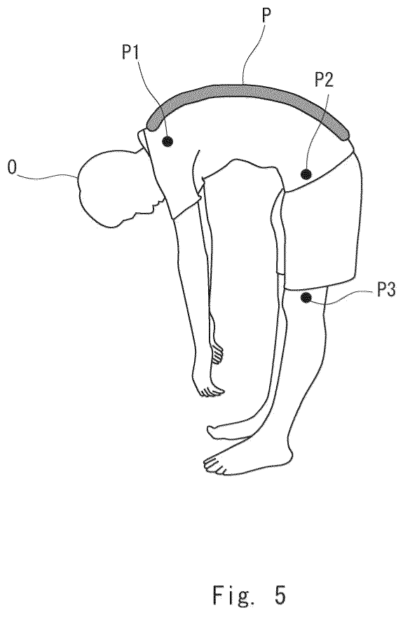

The spine extracting unit 103 extracts the spine on the image as a spine edge point cloud based on the image input from the image-capturing unit 101 and the position information of the key points input from the skeleton extracting unit 102. FIG. 5 illustrates an example of a spine edge point cloud P extracted by the spine extracting unit 103 from the image illustrated in FIG. 3. It should be noted that the details of the spine edge point cloud are as described in the first example embodiment, and therefore, the explanation thereabout is omitted.

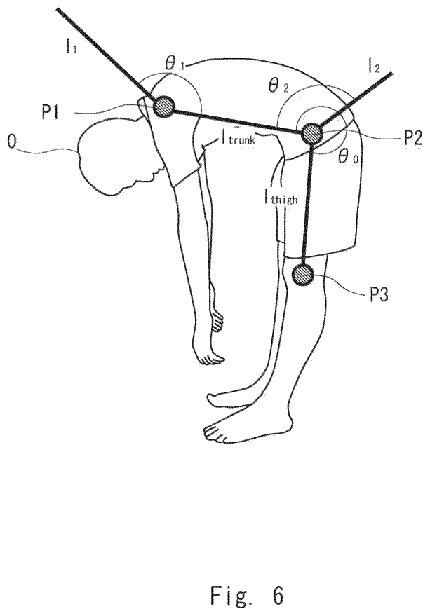

The processing of the spine extracting unit 103 is explained in detail below with reference to FIG. 6. First, the spine extracting unit 103 calculates the length of a line segment lₜᵣᵤₙₖ connecting the vertebral key point P1 and the hip joints key point P2, and the length of the line segment l_{thigh} connecting the hip joints key point P2 and the knee joints key point P3, and normalizes the size of the image input from the image-capturing unit 101. At this time, the vertebral key point P1, the hip joints key point P2, the knee joints key point P3, the length of the line segment lₜᵣᵤₙₖ, and the length of the line segment l_{thigh} are also converted to normalized values.

Next, the spine extracting unit 103 identifies the line of the back of the person O in the image based on the line segment 1ₜᵣᵤₙₖ and the line segment 1_{thigh}, and extracts a candidate edge point cloud that is a candidate for the spine edge point cloud P. Specifically, the spine extracting unit 103 specifies a bounding box that includes at least the back of the person O in the image based on the line segment lₜᵣᵤₙₖ and the line segment l_{thigh}. Next, the spine extracting unit 103 performs edge extraction processing on the image data within the bounding box, and extracts the candidate edge point cloud.

Next, the spine extracting unit 103 calculates the exterior angle θ₀ between the line segment lₜᵣᵤₙₖ and the line segment l_{thigh}. Next, the spine extracting unit 103 determines an angle θ₁ between the line segment l₁ that defines the head side of the spine and the line segment 1ₜᵣᵤₙₖ, and an angle θ₂ between the line segment l₂ that defines the tail side of the spine and the line segment lₜᵣᵤₙₖ, based on an exterior angle θ₀. Specifically, an angle table (not illustrated) that associates the exterior angle θ₀ with the angle θ₁ and the angle θ₂ is stored in advance in the storage unit 110, and the spine extracting unit 103 determines the angle θ₁ and the angle θ₂ based on the exterior angle θ₀ by referring to the angle table. In addition, an angle determination model (not illustrated) that has been machine-trained in advance using the angle table as training data is stored in the storage unit 110, and the spine extracting unit 103 may use the angle determination model to determine the angles θ₁ and θ₂ based on the exterior angle θ₀ .

Next, the spine extracting unit 103 determines, as a cranial end point of the spine edge point cloud P, an intersection point between the line segment l₁ and the candidate edge point cloud, and determines, as a caudal end point of the spine edge point cloud P, the intersection point between the line segment l₂ and the candidate edge point cloud. This determines the range of the candidate edge point clouds that becomes the spine edge point cloud P. In other words, the spine edge point cloud P is extracted.

Next, spine extracting unit 103 inputs the calculated exterior angle θ₀ to posture determining unit 104. In addition, the spine extracting unit 103 inputs the normalized vertebral key point P1, the hip joints key point P2, and the knee joints key point P3, and the extracted spine edge point cloud P to feature value calculating unit 105. In addition, the spine extracting unit 103 inputs the normalized image, the normalized key points P1, P2, P3, and the extracted spine edge point cloud P to the image generating unit 107.

The posture determining unit 104 determines the type of posture of the person O appearing in the image captured by the image-capturing unit 101 based on the exterior angle θ₀ input from the spine extracting unit 103. Specifically, a posture table (not illustrated) that associates the exterior angle θ₀ with the posture type is stored in advance in the storage unit 110, and the posture determining unit 104 determines the posture type based on the exterior angle θ₀ by referring to the posture table. Here, examples of posture types include bending forward, standing, bending backward, and the like. The posture determining unit 104 inputs the determined posture type to the state estimating unit 106.

The type of posture may be specified by the user through the input unit 109. The type of posture specified by the user may then be input to the state estimating unit 106.

The feature value calculating unit 105 calculates at least a feature value related to the spine based on the key points P1, P2, and P3 and the spine edge point cloud P input from the spine extracting unit 103. Specifically, the feature value calculating unit 105 calculates a spine curvature, a spine curvature angle, a hip joints angle, an upper thoracic curvature angle, a lower thoracic curvature angle, a lumbar curvature angle, and the like, as feature values based on the vertebral key point P1, the hip joints key point P2, the knee joints key point P3, and the spine edge point cloud P.

Specifically, the feature value calculating unit 105 calculates the spine curvature, i.e., a curvature of the spine, for all points included in the spine edge point cloud P by fitting an n-th order power function or a spline function to the spine edge point cloud P. For example, the feature value calculating unit 105 fits a cubic function to the spine edge point cloud P at each point included in the spine edge point cloud P to calculate the spine curvature at each point included in the spine edge point cloud P.

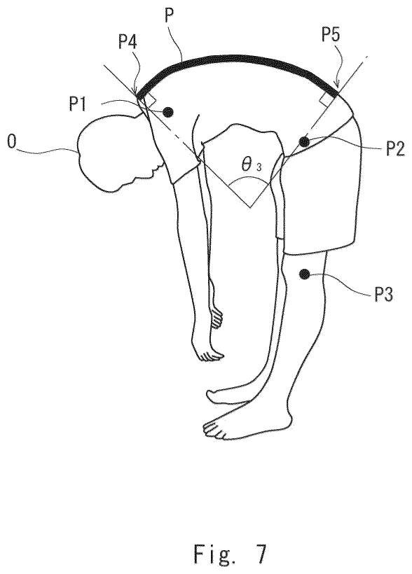

Next, the calculation process of the spine curvature angle by the feature value calculating unit 105 is described in detail with reference to FIG. 7. As illustrated in FIG. 7, the feature value calculating unit 105 calculates, as the spine curvature angle, the angle θ₃ between the normal to the spine edge point cloud P at the cranial end point P4 of the spine edge point cloud P and the normal to the spine edge point cloud P at the caudal end point P5 of the spine edge point cloud P.

Likewise, the feature value calculating unit 105 calculates the upper thoracic curvature angle, the lower thoracic curvature angle, and the lumbar curvature angle. Specifically, the positions of the upper thoracic vertebrae, the lower thoracic vertebrae, and the lumbar vertebrae in the spine are determined in advance as 0% to A %, A % to B %, and B % to C % of the spine from the cranial side of the spine, respectively. The feature value calculating unit 105 calculates, as the upper thoracic curvature angle, the angle between the normal to the spine edge point cloud P at the cranial end point of the upper thoracic vertebrae and the normal to the spine edge point cloud P at the caudal end point of the upper thoracic vertebrae.

Likewise, the feature value calculating unit 105 calculates, as the lower thoracic curvature angle, the angle between the normal to the spine edge point cloud P at the cranial end point of the lower thoracic vertebra and the normal to the spine edge point cloud P at the caudal end point of the lower thoracic vertebra.

The feature value calculating unit 105 calculates, the lumbar curvature angle, as the angle between the normal to the spine edge point cloud P at the cranial end point of the lumbar vertebrae and the normal to the spine edge point cloud P at the caudal end point of the lumbar vertebrae.

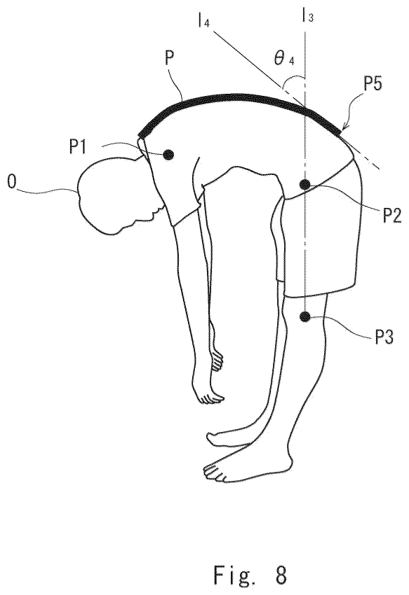

Next, with reference to FIG. 8, the calculation process of the hip joints angle by the feature value calculating unit 105 is explained in detail. The feature value calculating unit 105 calculates a tangent line l₄ to spine edge point cloud P at the caudal end point P5 of spine edge point cloud P. This tangent line l₄ represents the angle of the posterior surface of the sacrum. Next, the feature value calculating unit 105 calculates, as the hip joints angle, the angle θ₄ between the line segment l₃ connecting the hip joints key point P2 and the knee joints key point P3 and the tangent line l₄.

Then, the feature value calculating unit 105 inputs the calculated feature values, such as the spine curvature, the spine curvature angle, the hip joints angle, the upper thoracic curvature angle, the lower thoracic curvature angle, and lumbar curvature angle, to the state estimating unit 106.

The state estimating unit 106 estimates the state of at least the spine based on the feature values input from the feature value calculating unit 105. Specifically, the state estimating unit 106 estimates the states of the upper thoracic vertebrae, the lower thoracic vertebrae, the lumbar vertebrae, the lumbar-sacral junction (L5/S1), and the hip joints, based on the spine curvature, the spine curvature angle, the hip joints angle, the upper thoracic curvature angle, the lower thoracic curvature angle, the lumbar curvature angle, and the like, input from the feature value calculating unit 105.

Then, the state estimating unit 106 inputs the estimation results to the image generating unit 107.

For example, the state estimating unit 106 estimates the curvature of each part of the spine based on the spine curvature input from the feature value calculating unit 105. For example, in a case where the sign of the spine curvature at a certain point of the spine edge point cloud P is positive, the curvature of the spine at that point is convex toward the front side of the person O illustrated in the image captured by the image-capturing unit 101. In this case, in a case where the sign of the spine curvature at that point of the spine edge point cloud P is negative, the curvature of the spine at that point is convex toward the rear side of the person O. Therefore, the state estimating unit 106 estimates the curvature of each part of the spine based on the sign of the spine curvature. In other words, in a case where the sign of the spine curvature is positive, the state estimating unit 106 estimates that the curvature of the spine at that point is convex toward the front side of the person O. In a case where the sign of the spine curvature is negative, the state estimating unit 106 estimates that the curvature of the spine at that point is convex toward the rear of the person O.

Moreover, the state estimating unit 106 estimates the state of the spine, and the like, based on the type of posture input from the posture determining unit 104 and the feature value input from the feature value calculating unit 105. Specifically, the storage unit 110 stores the reference value list 112 in which the type of posture and the reference value for the feature value of the posture are associated with each other in advance. Then, the state estimating unit 106 estimates the state of the spine, and the like, by referring to the reference value list 112 based on the type of posture input from the posture determining unit 104 and the feature value input from the feature value calculating unit 105. In this case, the reference value is a value within the range that the feature value can take when the body part such as the spine is normal.

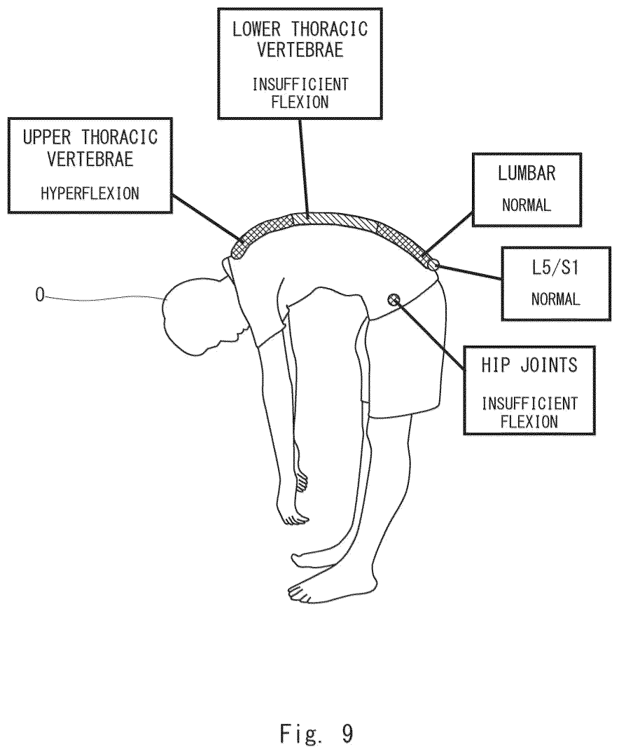

For example, as illustrated in FIG. 9, the state estimating unit 106 estimates the state of the upper thoracic spine as “hyperflexion”, by comparing the reference value of the upper thoracic curvature angle when the posture type is forward bending with the upper thoracic curvature angle input from the feature value calculating unit 105.

Likewise, the state estimating unit 106 estimates the state of the lower thoracic vertebrae as “insufficient flexion”, by comparing the reference value of the lower thoracic curvature angle when the posture type is forward bending with the lower thoracic curvature angle input from the feature value calculating unit 105.

Likewise, the state estimating unit 106 estimating the lumbar state as “normal”, by comparing the reference value of the lumbar curvature angle when the posture type is forward bending with the lumbar curvature angle input from the feature value calculating unit 105.

Likewise, the state estimating unit 106 estimates the state of the lumbar-sacral junction (L5/S1) as “normal”, by comparing the reference value of the lumbar curvature angle when the posture type is forward bending with the lumbar curvature angle input from the feature value calculating unit 105.

Likewise, the state estimating unit 106 estimates the state of the hip joints as “insufficient flexion”, by comparing the reference value of the hip joints angle when the posture type is forward bending with the hip joints angle input from the feature value calculating unit 105.

In addition, a machine-learned state estimation model (not illustrated) may be stored in advance in the storage unit 110, and the state estimating unit 106 may estimate the state of at least the spine using the state estimation model. Specifically, the storage unit 110 may store in advance training data in which the feature values related to at least the spine are associated with state labels such as “hyperflexion”, “normal”, or “insufficient flexion” as correct answer data. The storage unit 110 may store in advance a state estimation model that has been machine-learned using the training data.

The image generating unit 107 generates an estimation result display image to be displayed by the display unit 108, based on the normalized image, the normalized key points P1, P2, P3, the extracted spine edge point cloud P, which are input from the spine extracting unit 103, and the estimation result input from the state estimating unit 106.

In addition, the image generating unit 107 may generate a correction image for a user to correct the spine edge point cloud P based on the normalized image and extracted spine edge point cloud P input from the spine extracting unit 103.

Then, the image generating unit 107 inputs the generated image to the display unit 108.

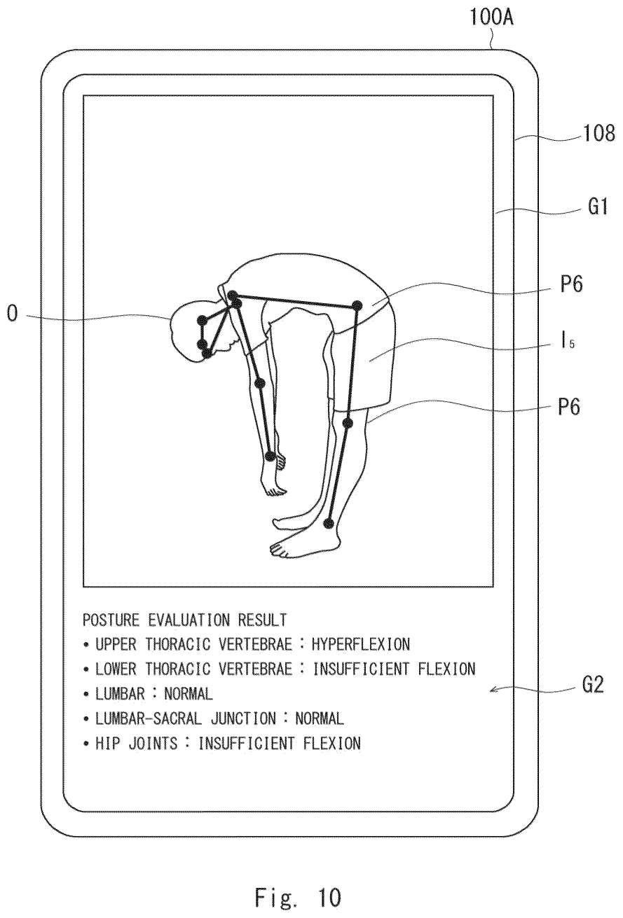

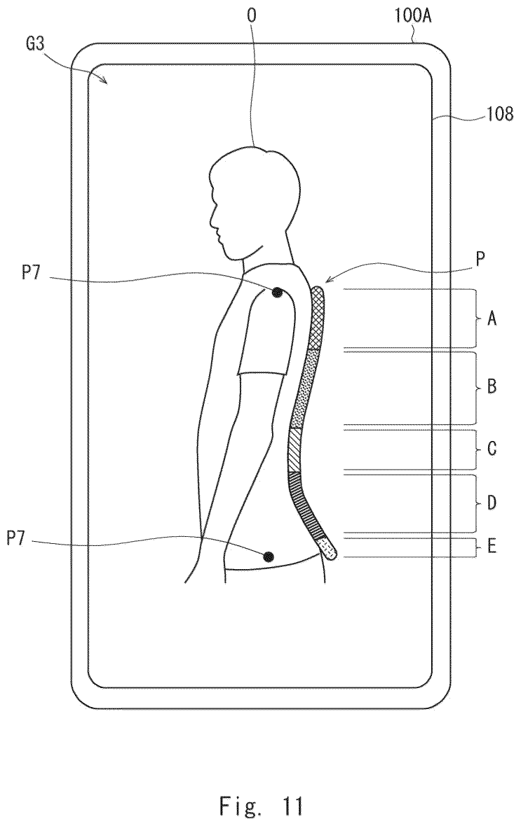

The display unit 108 displays the estimation result display image input from the image generating unit 107. The display unit 108 is constituted by various display means such as an LCD (Liquid Crystal Display) and an LED (Light Emitting Diode). FIG. 10 illustrates an example of an estimation result display image displayed on the display unit 108. FIG. 11 illustrates another example of an estimation result display image displayed on the display unit 108.

In the example illustrated in FIG. 10, an image portion G1 in which key points P6 and a line segment 15 connecting the key points P6 are superimposed on an image captured by the image-capturing unit 101 is displayed on the upper side of the display unit 108, and an image portion G2 illustrating the estimation result of the state estimating unit 106 is displayed on the lower side of display unit 108 of the posture evaluation apparatus 100A. Furthermore, in the image portion G2 illustrating the estimation result, estimation results other than “normal” may be displayed highlighted in bold, red, or the like.

In the example illustrated in FIG. 11, the display unit 108 of the posture evaluation apparatus 100A displays an image G3 in which key points P7 and the spine edge point cloud P, which is color-coded based on the estimation result of the state estimating unit 106, is superimposed on the image captured by the image-capturing unit 101. For example, on the display unit 108 illustrated in FIG. 11, each part A, B, C, D, and E of the spine edge point cloud P is displayed color-coded based on whether it is convex toward the front or toward the back of the person O illustrated in the image captured by the image-capturing unit 101. In addition, the color coding of each part A, B, C, D, and E of the spine edge point cloud P is performed based on the degree of protrusion (the magnitude of the spine curvature value) of each part A, B, C, D, and E.

The display unit 108 may also divide the spine edge point cloud P into portions, such as the upper thoracic vertebrae, the lower thoracic vertebrae, and the lumbar, and display each portion in a color according to the state of that portion.

Furthermore, the spine edge point cloud P may be colored using a gradation that gradually changes color.

The display unit 108 may also display the correction image input from the image generating unit 107. This allows, for example, in a case where the range of the spine edge point cloud P extracted by the spine extracting unit 103 is incorrect, the user to correct it by dragging the spine edge point cloud P displayed on the screen requiring correction.

The input unit 109 receives operation instructions from a user. The input unit 109 may be constituted by a keyboard or a touch panel display apparatus. The input unit 109 may be constituted by a keyboard or a touch panel connected to the main body of the posture evaluation apparatus 100A.

The storage unit 110 stores a reference value list 112, a skeleton database 113, a skeleton extraction model 114, and the like. The storage unit 110 may also include a non-volatile memory (e.g., ROM (Read Only Memory)) in which various programs and various data required for processing are fixedly stored. The storage unit 110 may also use an HDD (Hard Disk Drive) or an SSD. The storage unit 110 may also include a volatile memory (e.g., RAM (Random Access Memory)) used as a working area. The above programs may be read from a portable recording medium such as an optical disk or a semiconductor memory, or may be downloaded from a server apparatus on a network.

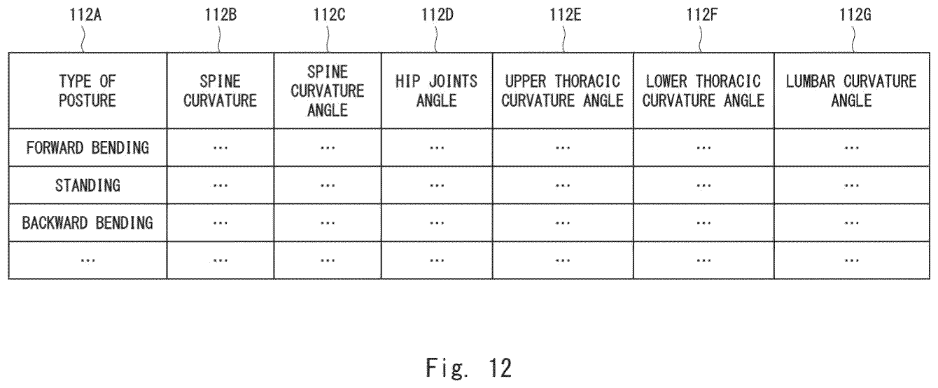

The reference value list 112 is a list in which the type of posture and the reference value for the feature value of the posture are associated with each other in advance. FIG. 12 illustrates an example of a data structure of the reference value list 112. As illustrated in FIG. 12, the reference value list 112 is a list that lists, in association with each other, a type of posture 112A, a reference value 112B of the spine curvature at that posture, a reference value 112C of the spine curvature angle, a reference value 112D of the hip joints angle, a reference value 112E of the upper thoracic curvature angle, a reference value 112F of the lower thoracic curvature angle, a reference value 112G of the lumbar curvature angle.

The skeleton database 113 is a database in which each of multiple images acquired by imaging the side surface of the body is associated with the position information of the key points as correct labels.

The skeleton extraction model 114 is a machine learning model that extracts position information of key points from an image acquired by imaging the side surface of the body. In other words, the skeleton extraction model 114 is a machine learning model that uses an image acquired by imaging the side surface of the body as input, and infers and outputs position information of key points. It should be noted that, in this specification, machine learning may be deep learning, but is not particularly limited thereto.

The communication unit 111 communicates with external servers and other terminal apparatuses. The communication unit 111 may be equipped with an antenna (not illustrated) for wireless communication, or may be equipped with an interface such as a NIC (Network Interface Card) for wired communication.

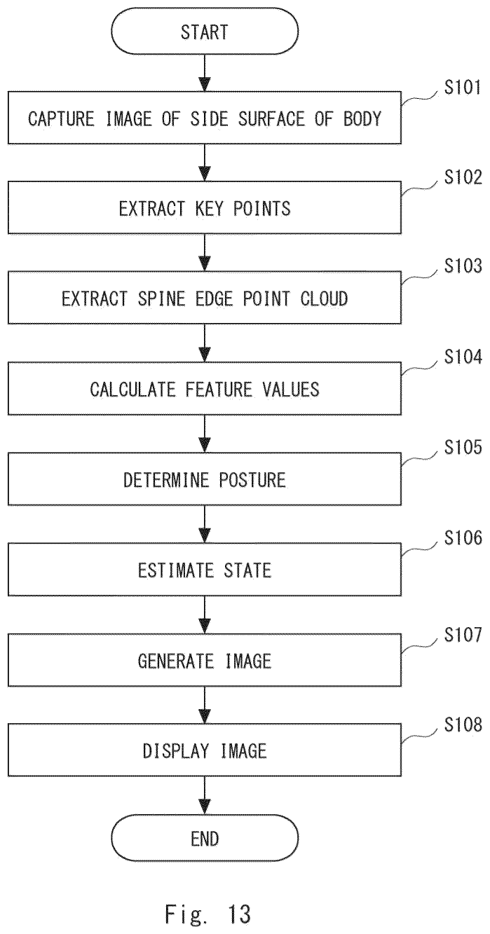

Next, the posture evaluation method according to the second example embodiment is described with reference to FIG. 13. First, the image-capturing unit 101 captures an image of the side surface of the body (step S101), and inputs the acquired image to the skeleton extracting unit 102 and the spine extracting unit 103.

Next, the skeleton extracting unit 102 extracts position information of key points from the image captured by the image-capturing unit 101 in step S101 (step S102), and inputs the position information of the extracted key points to the spine extracting unit 103. For example, the skeleton extracting unit 102 extracts the position information of the vertebrae key point P1, the hip joints key point P2, and the knee joints key point P3.

Next, the spine extracting unit 103 extracts a spine edge point cloud based on the image captured by the image-capturing unit 101 in step S101 and the position information of the key points P1, P2, and P3 extracted by the skeleton extracting unit 102 in step S102 (step S103). Specifically, the spine extracting unit 103 normalizes the image captured by the image-capturing unit 101, calculates the exterior angle θ₀ between the line segment lₜᵣᵤₙₖ and the line segment l_{thigh}, and extracts the spine edge point cloud P. Then, the spine extracting unit 103 inputs the calculated exterior angle θ₀ to the posture determining unit 104. The spine extracting unit 103 also inputs the key points P1, P2, and P3 and the spine edge point cloud P to the feature value calculating unit 105. In addition, the spine extracting unit 103 inputs the normalized image, the key points P1, P2, P3, and the spine edge point cloud P to the image generating unit 107.

Next, the feature value calculating unit 105 calculates the spine curvature, the spine curvature angle, the hip joints angle, the upper thoracic curvature angle, the lower thoracic curvature angle, the lumbar curvature angle, and the like, as feature values based on the key points P1, P2, P3 and the spine edge point cloud P, which are input from the spine extracting unit 103 (step S104). Then, the feature value calculating unit 105 inputs the calculated spine curvature, the spine curvature angle, the hip joints angle, the upper thoracic curvature angle, the lower thoracic curvature angle, the lumbar curvature angle, and the like, to the state estimating unit 106.

The posture determining unit 104 determines the type of posture of the person O appearing in the image captured by the image-capturing unit 101, based on the exterior angle θ₀ input from the spine extracting unit 103 (step S105). The posture determining unit 104 then inputs the determined type of posture to the state estimating unit 106.

Next, the state estimating unit 106 estimates the states of the upper thoracic vertebrae, the lower thoracic vertebrae, the lumbar, the lumbar-sacral junction (L5/S1), and the hip joints, based on the type of posture input from the posture determining unit 104 and the spine curvature, the spine curvature angle, the hip joints angle, the upper thoracic curvature angle, the lower thoracic curvature angle, the lumbar curvature angle, and the like, which are input from the feature value calculating unit 105 (step S106). Then, the state estimating unit 106 inputs the estimation result to the image generating unit 107.

Next, the image generating unit 107 generates an image to be displayed on the display unit 108 based on the image captured in step S101 and the state of the upper thoracic vertebrae, the lower thoracic vertebrae, the lumbar, the lumbar-sacral junction (L5/S1), and the hip joints estimated in step S106 (step S107). The image generated by the image generating unit 107 is then input to the display unit 108.

Next, the display unit 108 displays the image generated in step S107 (step S108), and the process ends.

The key points P1, P2, and P3 may be specified by the user operating the input unit 109 when the image captured by the image-capturing unit 101 is displayed on the display unit 108. In this case, the processing of step S102 may be omitted.

After the processing of step S103 and before the processing of step S104, a correction image illustrating the spine edge point cloud P extracted in step S103 may be displayed on the display unit 108, and the user may correct the spine edge point cloud P displayed on the screen requiring correction by dragging it.

The order of the processing of steps S104 and S105 may be reversed, and the processing of steps S104 and S105 may be performed simultaneously.

Before the processing of step S106, the user may specify the type of posture by operating the input unit 109. In this case, the processing of step S105 may be omitted.

According to this second example embodiment, the posture evaluation apparatus 100A that can evaluate the posture with a high accuracy at a relatively low cost can be provided. Specifically, the spine extracting unit 103 extracts the spine edge point cloud P representing the spine shape on the image, and the feature value calculating unit 105 calculates the spine curvature, the spine curvature angle, the hip joints angle, the upper thoracic curvature angle, the lower thoracic curvature angle, and the lumbar curvature angle as feature values, based on the key points P1, P2, and P3 on the image and the spine edge point cloud P. Then, the state estimating unit 106 estimates the states of the upper thoracic vertebrae, the lower thoracic vertebrae, the lumbar, the lumbar-lumbar-sacral junction (L5/S1), and the hip joints, based on the feature values. In other words, the posture can be evaluated based on the spine shape on the image, and accordingly, the posture can be evaluated with a high accuracy. Furthermore, the posture evaluation can be performed based on the spine shape without using expensive specialized equipment, so the posture can be evaluated at a relatively low cost. Therefore, the posture evaluation apparatus 100A that can evaluate the posture with a high accuracy at a relatively low cost can be provided.

In addition, the skeleton extracting unit 102 extracts the key points P1, P2, and P3 from the image captured by the image-capturing unit 101. This eliminates the need for the user to specify the key points P1, P2, and P3 on the image.

The skeleton extracting unit 102 may also use a trained skeleton extraction model 114 to extract key points expressed in three-dimensional coordinates defined in the z direction, i.e., the depth direction, in addition to the x direction, i.e., the left-right or horizontal direction, and the y direction, i.e., the up-down or vertical direction, of the two-dimensional image captured by the image-capturing unit 101. This enables more precise posture evaluation.

Also, the state estimating unit 106 estimates the state of the spine, and the like, based on the type of posture and the feature value by referring to the reference value list 112. In this case, the reference value is the range of values that the feature value can take when the body part, such as the spine, is normal. Therefore, the state estimating unit 106 can estimate whether the body part, such as the spine, is normal or not.

In addition, the posture determining unit 104 determines the type of posture of the person O to perform posture evaluation based on the key points extracted by the skeleton extracting unit 102. Therefore, the user does not need to specify the type of posture to perform posture evaluation.

In addition, the display unit 108 displays an estimation result display image that illustrates the image captured by the image-capturing unit 101 and the estimation result by the state estimating unit 106. This allows the user to visually find the state of posture.

In addition, the display unit 108 displays the state estimated by the state estimating unit 106 by coloring the spine edge point cloud P. This allows the user to visually grasp the state of posture.

### Third Example Embodiment

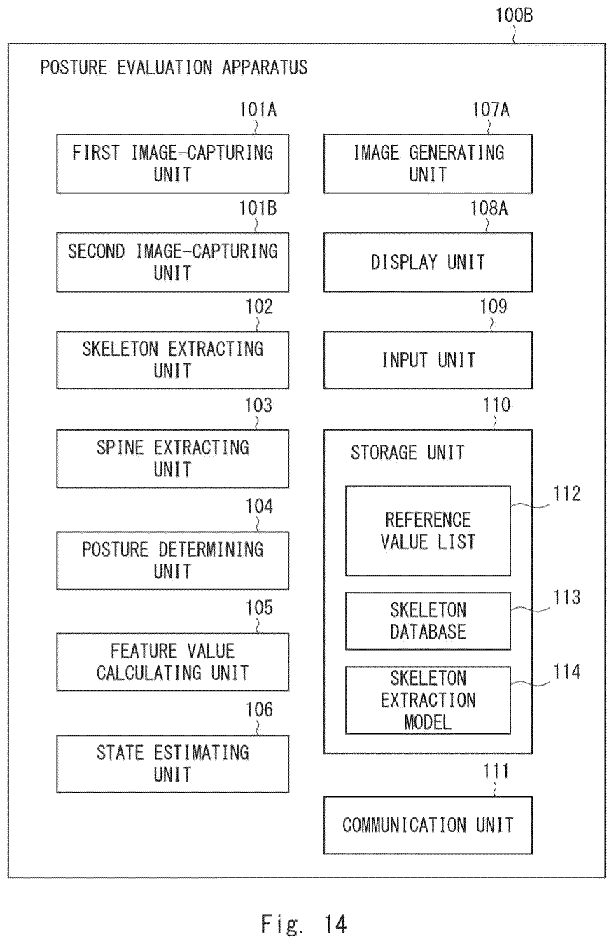

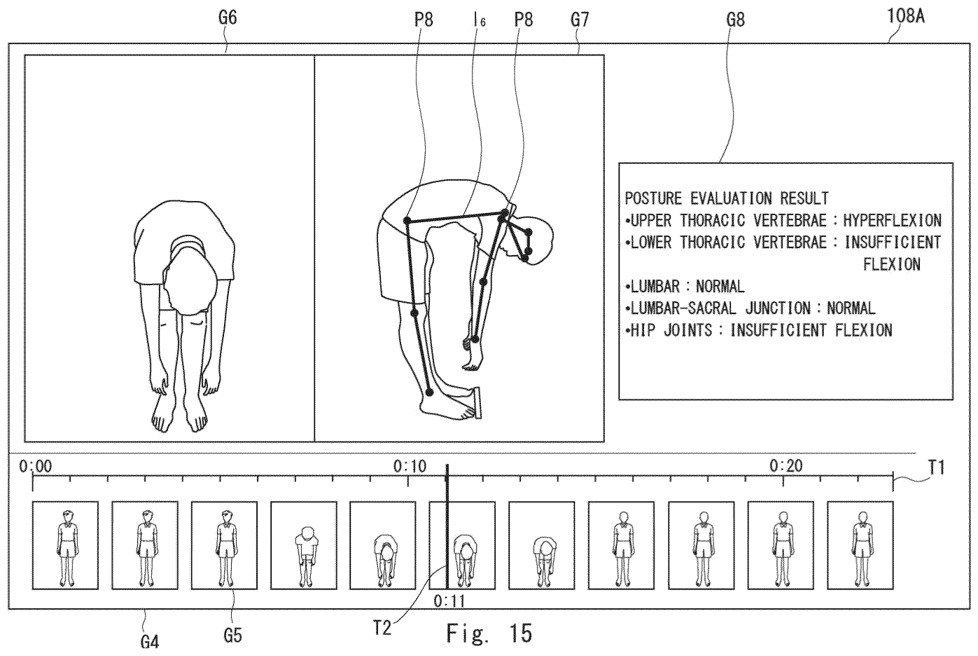

Next, a posture evaluation apparatus 100B according to this third example embodiment is described with reference to FIG. 14. FIG. 14 is a block diagram illustrating a configuration of the posture evaluation apparatus 100B according to this third example embodiment. The posture evaluation apparatus 100B according to the third example embodiment differs from the posture evaluation apparatus 100A according to the second example embodiment in that the posture evaluation apparatus 100B is equipped with a first image-capturing unit 101A and a second image-capturing unit 101B. In addition, as illustrated in FIG. 15, the image generated by an image generating unit 107A, i.e., an image displayed on a display unit 108A, are also different. Therefore, among the constituent elements of the posture evaluation apparatus 100B according to the third example embodiment, the same constituent elements as those of the posture evaluation apparatus 100A according to the second example embodiment are denoted with the same reference numerals and description thereabout is omitted.

The first image-capturing unit 101A captures an image of the side surface of the body of the subject person, similar to the image-capturing unit 101 according to the second example embodiment. The first image-capturing unit 101A inputs the captured image to the skeleton extracting unit 102, the spine extracting unit 103, and the image generating unit 107A.

Similar to the image-capturing unit 101 according to the second example embodiment, the first image-capturing unit 101A may capture a video of the side surface of the body of the subject person to acquire an image. In this case, the user operates the input unit 109 to specify the time point at which posture evaluation is to be performed in an image selection area G4 (see FIG. 15) displayed on the display unit 108A. The image at the time point specified by the user is then input to the skeleton extracting unit 102 and spine extracting unit 103. In this third example embodiment, an example is described in which the first image-capturing unit 101A captures a video of the side surface of the subject person.

The second image-capturing unit 101B captures another surface of the body of the subject person simultaneously with the first image-capturing unit 101A. In this case, the another surface of the body of the subject person may be any surface other than the side surface of the body of the subject person. The second image-capturing unit 101B inputs the captured image to the image generating unit 107A.

The second image-capturing unit 101B may also capture a video of the another surface of the body of the subject person to acquire an image. In this third example embodiment, an example is described in which the second image-capturing unit 101B captures a video of the front surface of the body of the subject person.

Based on the image input from the second image-capturing unit 101B, the image generating unit 107A generates an image selection area G4 to be displayed by the display unit 108A. As illustrated in FIG. 15, the image selection area G4 is an image area in which multiple thumbnail images G5 of the video input from the second image-capturing unit 101B are arranged along a time scale T1, and a designation bar T2 for designating the time point at which posture evaluation is performed is displayed movably along the time scale T1.

The image generating unit 107A also generates a front surface image area G6 that illustrates a front surface image that displays the image at the time point designated by the user in the image selection area G4 displayed on the display unit 108A.

The image generating unit 107A also generates a side surface image portion G7 in which a key point P8 and a line segment 16 connecting the key point P8 are superimposed on the image, based on the normalized image and the key point input from the spine extracting unit 103.

In addition, the image generating unit 107A generates a result image area G8 indicating the estimation result of state estimating unit 106 based on the estimation result input from the state estimating unit 106.

The image generating unit 107A then inputs the generated image selection area G4, the front surface image area G6, the side surface image area G7, and the result image area G8 to the display unit 108A.

The display unit 108A displays the image selection area G4, the front surface image area G6, the side surface image area G7, and the result image area

G8, which are input from the image generating unit 107. FIG. 15 illustrates an example of an image displayed on the display unit 108A.

In the example illustrated in FIG. 15, the image selection area G4 is displayed below the display unit 108A of the posture evaluation apparatus 100B, the front surface image area G6 is displayed on the upper left side of the display unit 108A, the side surface image area G7 is displayed in the upper center of the display unit 108A, and the result image area G8 is displayed on the upper right side of the display unit 108A. In the example shown in FIG. 15, the designation bar T2 is moved by the user in the image selection area G4, and 0 minutes 11 seconds is designated as the time point for the posture evaluation.

According to this third example embodiment, the user can see the front surface image area G6 displayed on the display unit 108A to find information about the posture that cannot be acquired from the side surface of the body. For example, the user can check from the front surface image area G6 whether the left and right sides of the body are moving evenly.

### Fourth Example Embodiment

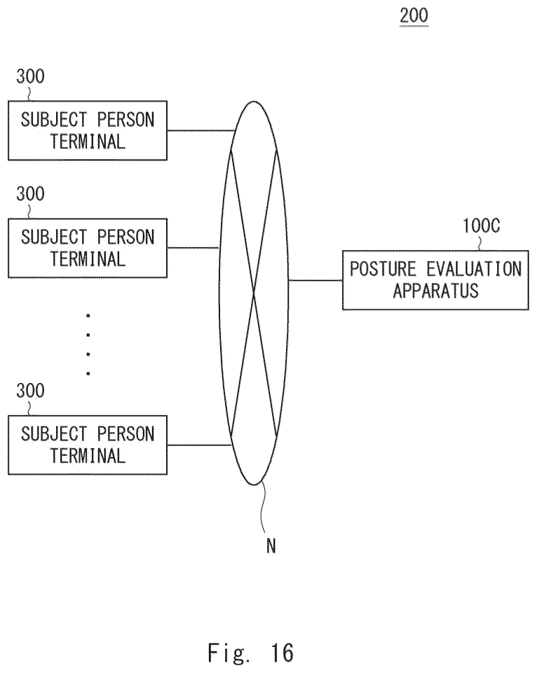

Next, a posture evaluation system 200 according to the fourth example embodiment is described with reference to FIG. 16. FIG. 16 is a diagram illustrating a configuration of the posture evaluation system 200 according to the fourth example embodiment. As illustrated in FIG. 16, the posture evaluation system 200 includes a posture evaluation apparatus 100C and a subject person terminal 300 capable of communicating with the posture evaluation apparatus 100C. The posture evaluation apparatus 100C and the subject person terminal 300 are capable of communicating via a network N. Also, as illustrated in FIG. 16, one or more subject person terminals 300, . . . may be capable of communicating with the posture evaluation apparatus 100C.

Also, the subject person terminal 300 is a smartphone, tablet terminal, personal computer, and the like, owned by the subject person.

The posture evaluation apparatus 100C according to the fourth example embodiment acquires an image of the side surface of the body of the subject person from the subject person terminal 300. Therefore, the posture evaluation apparatus 100C differs from the posture evaluation apparatus 100A according to the second example embodiment in that the image-capturing unit 101 may be omitted.

In addition, an estimation result display image generated by the image generating unit 107 of the posture evaluation apparatus 100C may be transmitted to the subject person terminal 300 and displayed on a display unit (not illustrated) of the subject person terminal 300.

The subject person terminal 300 includes an image-capturing unit (not illustrated) that captures an image of the side surface of the body of the subject person. The subject person terminal 300 transmits the image to the posture evaluation apparatus 100C.

### Other Embodiments

Next, a posture evaluation method according to other embodiments is briefly described. The posture evaluation system according to the other embodiments is a modified example of the posture evaluation system 200. The posture evaluation apparatus 100C according to the other embodiments acquires an image of the side surface and another surface of the body of the subject person simultaneously from the subject person terminal 300. In this case, the posture evaluation apparatus 100C differs from the posture evaluation apparatus 100B according to the third example embodiment in that the first image-capturing unit 101A and the second image-capturing unit 101B may be omitted.

In addition, the image selection area G4, the front surface image area G6, the side surface image area G7, and the result image area G8 generated by the image generating unit 107A of the posture evaluation apparatus 100C may be transmitted to the subject person terminal 300 and displayed on a display unit (not illustrated) of the subject person terminal 300.

The subject person terminal 300 includes a first image-capturing unit (not illustrated) that captures an image of a side surface of the body of the subject person, and a second image-capturing unit (not illustrated) that captures an image of another surface of the body of the subject person simultaneously with the first image-capturing unit. The subject person terminal 300 transmits an image of the side surface of the body and an image of the another surface of the body to the posture evaluation apparatus 100C.

According to this fourth example embodiment and other embodiments, at least the side surface of the body of the subject person is imaged by the subject person terminal 300, and the acquired image is transmitted to the posture evaluation apparatus 100C via the network N, and posture evaluation can be performed by the posture evaluation apparatus 100C. Therefore, for example, even if the subject person and the evaluator are in different locations, the evaluator can remotely evaluate the posture of the subject person. The posture evaluation system 200 according to this fourth example embodiment or other embodiments is particularly advantageous in situations such as remote therapy and remote training.

In the above-mentioned embodiments, the present disclosure has been described as a hardware configuration, but the present disclosure is not limited thereto. The present disclosure can also be realized by making a CPU (Central Processing Unit) execute a computer program to perform the processing steps illustrated in the flowchart of FIG. 13 and the processing steps described in the other embodiments.

In the above examples, the program includes instructions (or software code) that, when loaded into a computer, cause the computer to perform one or more functions described in the example embodiments. The program may be stored on a non-transitory computer-readable medium or tangible storage medium. By way of example and not limitation, the computer-readable medium or tangible storage medium includes random-access memory (RAM), read-only memory (ROM), flash memory, solid-state drive (SSD), or other memory technology, CD-ROM, digital versatile disc (DVD), Blu-ray disc, or other optical disk storage, magnetic cassette, magnetic tape, magnetic disk storage, or other magnetic storage device. The program may be transmitted on a transitory computer-readable medium or communication medium. By way of example and not limitation, the transitory computer-readable medium or communication medium includes electrical, optical, acoustic, or other form of propagated signal.

Although the present invention has been described above with reference to the embodiments, the present invention is not limited thereto. Various modifications that can be understood by a person skilled in the art can be made to the configuration and details of the present invention within the scope of the invention. For example, in a case where a subject person is wearing clothes that do not show the body lines, the spine extracting unit 103 cannot extract the spine edge point cloud P by using the edge point cloud extracted by the edge extraction process as a candidate edge point cloud. Therefore, the spine extracting unit 103 may perform estimation processing of the candidate edge point cloud based on the edge point cloud, the vertebrae key point P1, the hip joints key point P2, and the knee joints key point P3, which are extracted by the edge extraction process.

Some or all of the above-described example embodiments may be described as in the Supplementary Notes below, but are not limited thereto.

### Supplementary Note 1

A posture evaluation apparatus including:
spine extracting means for extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
feature value calculating means for calculating a feature value about at least a spine, based on the position information and the spine edge point cloud; and
state estimating means for estimating a state of at least the spine, based on the feature value.

### Supplementary Note 2

The posture evaluation apparatus according to Supplementary Note 1, further including skeleton extracting means for extracting the position information from the image.

### Supplementary Note 3

The posture evaluation apparatus according to Supplementary Note 1 or 2, further including storage means for storing, in association with each other, a type of a posture of the body and a reference value of the feature value for the type of the posture,
wherein the state estimating means estimates a state of at least the spine, based on the type of the posture, the feature value calculated by the feature value calculating means, and the reference value.

### Supplementary Note 4

The posture evaluation apparatus according to Supplementary Note 3, further including posture determining means for determining a type of the posture, based on the position information.

### Supplementary Note 5

The posture evaluation apparatus according to any one of Supplementary Notes 1 to 4, further including display means for displaying the image and the state estimated by the state estimating means.

### Supplementary Note 6

The posture evaluation apparatus according to Supplementary Note 5, wherein the display means displays the state by color-coding the spine edge point cloud.

### Supplementary Note 7

The posture evaluation apparatus according to Supplementary Note 5 or 6, wherein, in a case where the spine extracting means extracts the spine edge point cloud, the display means displays, together with the image, the spine edge point cloud in such a way that the spine edge point cloud can be modified by a user.

### Supplementary Note 8

The posture evaluation apparatus according to any one of Supplementary Notes 5 to 7, further including:
first image-capturing means for imaging the side surface of the body; and
second image-capturing means for imaging another surface of the body, simultaneously with the first image-capturing means,
wherein the display means displays an image of the side surface of the body imaged by the first image-capturing means and an image of another surface of the body imaged by the second image-capturing means.

### Supplementary Note 9

A posture evaluation system including:
a posture evaluation apparatus; and
a subject person terminal that can communicate with the posture evaluation apparatus,
wherein the posture evaluation apparatus includes:
   spine extracting means for extracting, based on an image of a side surface of a body of a subject person being acquired by the subject person terminal and position information about at least a cervical vertebrae, hip joints, and knee joints of the body on the image, a spine edge point cloud constituted of a predetermined number of points representing a spine shape on the image;
   feature value calculating means for calculating a feature value about at least a spine, based on the position information and the spine edge point cloud; and
   state estimating means for estimating a state of at least the spine, based on the feature value.

### Supplementary Note 10

The posture evaluation system according to Supplementary Note 9, wherein the posture evaluation apparatus includes skeleton extracting means for extracting the position information from the image.

### Supplementary Note 11

The posture evaluation system according to Supplementary Note 9 or 10, wherein
the posture evaluation apparatus includes storage means for storing, in association with each other, a type of a posture of the body and a reference value of the feature value for the type of the posture, and
the state estimating means estimates a state of at least the spine, based on the type of the posture, the feature value calculated by the feature value calculating means, and the reference value.

### Supplementary Note 12

The posture evaluation system according to Supplementary Note 11, wherein the posture evaluation apparatus includes posture determining means for determining a type of the posture, based on the position information.

### Supplementary Note 13

The posture evaluation system according to any one of Supplementary Notes 9 to 12, wherein the posture evaluation apparatus includes display means for displaying the image and the state estimated by the state estimating means.

### Supplementary Note 14

The posture evaluation system according to Supplementary Note 13, wherein the display means displays the state by color-coding the spine edge point cloud.

### Supplementary Note 15

The posture evaluation system according to Supplementary Note 13 or 14, wherein, in a case where the spine extracting means extracts the spine edge point cloud, the display means displays, together with the image, the spine edge point cloud in such a way that the spine edge point cloud can be modified by a user.

### Supplementary Note 16

The posture evaluation system according to any one of Supplementary Notes 13 to 15, wherein
the posture evaluation apparatus includes:
first image-capturing means for imaging the side surface of the body; and
second image-capturing means for imaging another surface of the body, simultaneously with the first image-capturing means, and
the display means displays an image of the side surface of the body imaged by the first image-capturing means and an image of another surface of the body imaged by the second image-capturing means.

### Supplementary Note 17

A posture evaluation method including, by a posture evaluation apparatus:
extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
calculating a feature value about at least a spine, based on the position information and the spine edge point cloud; and
estimating a state of at least the spine, based on the feature value.

### Supplementary Note 18

The posture evaluation method according to Supplementary Note 17, wherein the posture evaluation apparatus extracts the position information from the image.

### Supplementary Note 19

The posture evaluation method according to Supplementary Note 17 or 18, wherein the posture evaluation apparatus stores, in association with each other, a type of a posture of the body and a reference value of the feature value for the type of the posture, and the posture evaluation apparatus estimates a state of at least the spine, based on the type of the posture, the feature value, and the reference value.

### Supplementary Note 20

The posture evaluation method according to Supplementary Note 19, wherein the posture evaluation apparatus determines a type of the posture, based on the position information.

### Supplementary Note 21

The posture evaluation method according to any one of Supplementary Notes 17 to 20, wherein the posture evaluation apparatus displays the image and the state.

### Supplementary Note 22

The posture evaluation method according to any one of Supplementary Notes 17 to 21, wherein the posture evaluation apparatus displays the state by color-coding the spine edge point cloud.

### Supplementary Note 23

The posture evaluation method according to any one of Supplementary Notes 17 to 22, wherein, in a case where the spine edge point cloud is extracted, the posture evaluation apparatus displays, together with the image, the spine edge point cloud in such a way that the spine edge point cloud can be modified by a user.

### Supplementary Note 24

The posture evaluation method according to any one of Supplementary Notes 17 to 23, wherein the posture evaluation apparatus images another surface of the body, simultaneously with imaging of the side surface of the body, and displays an image of the side surface of the body being imaged and an image of another surface of the body being imaged.

### Supplementary Note 25

A non-transitory computer-readable medium configured to store a program causing a posture evaluation apparatus to execute:
processing of extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
processing of calculating a feature value about at least a spine, based on the position information and the spine edge point cloud; and
processing of estimating a state of at least the spine, based on the feature value.

### Supplementary Note 26

The non-transitory computer-readable medium according to Supplementary Note 25, storing the program causing the posture evaluation apparatus to execute processing of extracting the position information from the image.

### Supplementary Note 27

The non-transitory computer-readable medium according to Supplementary Note 25 or 26, storing the program causing the posture evaluation apparatus to execute:
processing of storing, in association with each other, a type of a posture of the body and a reference value of the feature value for the type of the posture; and
processing of estimating a state of at least the spine, based on the type of the posture, the feature value, and the reference value.

### Supplementary Note 28

The non-transitory computer-readable medium according to Supplementary Note 27, storing the program causing the posture evaluation apparatus to execute processing of determining a type of the posture, based on the position information.

### Supplementary Note 29

The non-transitory computer-readable medium according to any one of Supplementary Notes 25 to 28, storing the program causing the posture evaluation apparatus to execute processing of displaying the image and the state.

### Supplementary Note 30

The non-transitory computer-readable medium according to any one of Supplementary Notes 25 to 29, storing the program causing the posture evaluation apparatus to execute processing of displaying the state by color-coding the spine edge point cloud.

### Supplementary Note 31

The non-transitory computer-readable medium according to any one of Supplementary Notes 25 to 30, storing the program causing the posture evaluation apparatus to execute processing of, in a case where the spine edge point cloud is extracted, displaying, together with the image, the spine edge point cloud in such a way that the spine edge point cloud can be modified by a user.

### Supplementary Note 32

The non-transitory computer-readable medium according to any one of Supplementary Notes 25 to 31, storing the program causing the posture evaluation apparatus to execute
processing of imaging another surface of the body, simultaneously with imaging of the side surface of the body, and
processing of displaying an image of the side surface of the body being imaged and an image of another surface of the body being imaged.

This application claims priority based on Japanese Patent Application No. 2022-058198, filed on Mar. 31, 2022, the disclosure of which is incorporated herein in its entirety.

### INDUSTRIAL APPLICABILITY

A posture evaluation apparatus, a posture evaluation system, a posture evaluation method, and a non-transitory computer-readable medium that are able to evaluate a posture with high accuracy at a relatively low cost can be provided.

### Reference Signs List

100, 100A, 100B, 100C POSTURE EVALUATION APPARATUS
101 IMAGE-CAPTURING UNIT (IMAGE-CAPTURING MEANS)
101A FIRST IMAGE-CAPTURING UNIT (FIRST IMAGE-CAPTURING MEANS)
101B SECOND IMAGE-CAPTURING UNIT (SECOND IMAGE-CAPTURING MEANS)
102 SKELETON EXTRACTING UNIT (SKELETON EXTRACTING MEANS)
103 SPINE EXTRACTING UNIT (SPINE EXTRACTING MEANS)
104 POSTURE DETERMINING UNIT (POSTURE DETERMINING MEANS)
105 FEATURE VALUE CALCULATING UNIT (FEATURE VALUE CALCULATING MEANS)
106 STATE ESTIMATING UNIT (STATE ESTIMATING MEANS)
107, 107A IMAGE GENERATING UNIT
108, 108A DISPLAY UNIT (DISPLAY MEANS)
109 INPUT UNIT
110 STORAGE UNIT (STORAGE MEANS)
111 COMMUNICATION UNIT
112 REFERENCE VALUE LIST
113 SKELETON DB (SKELETON DATABASE)
114 SKELETON EXTRACTION MODEL
200 POSTURE EVALUATION SYSTEM
300 SUBJECT PERSON TERMINAL

1. A posture evaluation apparatus comprising:
a memory storing instructions; and
one or more processors configured to execute the instructions to:
extract, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
calculate a feature value about at least a spine, based on the position information and the spine edge point cloud; and
estimate a state of at least the spine, based on the feature value.

2. The posture evaluation apparatus according to claim 1, the one or more processors configured to execute the instructions to:
extract the position information from the image.

3. The posture evaluation apparatus according to claim 1, the one or more processors configured to execute the instructions to:
store, in association with each other, a type of a posture of the body and a reference value of the feature value for the type of the posture,
wherein the one or more processors estimate a state of at least the spine, based on the type of the posture, the feature value calculated by the feature value calculating means, and the reference value.

4. The posture evaluation apparatus according to claim 3, the one or more processors configured to execute the instructions to:
determine a type of the posture, based on the position information.

5. The posture evaluation apparatus according to claim 1, the one or more processors configured to execute the instructions to:
display the image and the state estimated.

6. The posture evaluation apparatus according to claim 5, wherein the one or more processors display the state by color-coding the spine edge point cloud.

7. The posture evaluation apparatus according to claim 5, wherein, in a case where the one or more processors extract the spine edge point cloud, the one or more processors display, together with the image, the spine edge point cloud in such a way that the spine edge point cloud can be modified by a user.

8. The posture evaluation apparatus according to claim 5, the one or more processors configured to execute the instructions to:
capture an image of the side surface of the body; and
capture an image of another surface of the body, simultaneously with capturing the image of the side surface of the body,
wherein the one or more processors display the image of the side surface of the body and the image of another surface of the body.

9-16. (canceled)

17. A posture evaluation method comprising, by a posture evaluation apparatus:
extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
calculating a feature value about at least a spine, based on the position information and the spine edge point cloud; and
estimating a state of at least the spine, based on the feature value.

18. The posture evaluation method according to claim 17, wherein the posture evaluation apparatus extracts the position information from the image.

19. The posture evaluation method according to claim 17, wherein the posture evaluation apparatus stores, in association with each other, a type of a posture of the body and a reference value of the feature value for the type of the posture, and the posture evaluation apparatus estimates a state of at least the spine, based on the type of the posture, the feature value, and the reference value.

20. The posture evaluation method according to claim 19, wherein the posture evaluation apparatus determines a type of the posture, based on the position information.

21. The posture evaluation method according to claim 17, wherein the posture evaluation apparatus displays the image and the state.

22. The posture evaluation method according to claim 17, wherein the posture evaluation apparatus displays the state by color-coding the spine edge point cloud.

23-24. (canceled)

25. A non-transitory computer-readable medium configured to store a program causing a posture evaluation apparatus to execute:
processing of extracting, based on an image acquired by imaging a side surface of a body of a subject person and position information about at least a cervical vertebrae, hip joints, and knee joints of the body on the image, a spine edge point cloud constituted of a predetermined number of points representing a spine shape on the image;
processing of calculating a feature value about at least a spine, based on the position information and the spine edge point cloud; and
processing of estimating a state of at least the spine, based on the feature value.

26. The non-transitory computer-readable medium according to claim 25, storing the program causing the posture evaluation apparatus to execute processing of extracting the position information from the image.

27. The non-transitory computer-readable medium according to claim 25, storing the program causing the posture evaluation apparatus to execute:
processing of storing, in association with each other, a type of a posture of the body and a reference value of the feature value for the type of the posture; and
processing of estimating a state of at least the spine, based on the type of the posture, the feature value, and the reference value.

28. The non-transitory computer-readable medium according to claim 27, storing the program causing the posture evaluation apparatus to execute processing of determining a type of the posture, based on the position information.

29. The non-transitory computer-readable medium according to claim 25, storing the program causing the posture evaluation apparatus to execute processing of displaying the image and the state.

30. The non-transitory computer-readable medium according to claim 25, storing the program causing the posture evaluation apparatus to execute processing of displaying the state by color-coding the spine edge point cloud.

31-32. (canceled)
